(19) United States
(12) Patent Application Publication
(10) Pub. No.: US 2021/0128486 A1
(43) Pub. Date: May 6, 2021

(54) NANOPARTICLE STRUCTURE

(71) Applicants: Seoul National University R&DB Foundation, Seoul (KR); INSTITUTE FOR BASIC SCIENCE, Daejeon (KR)

(72) Inventors: Taeghwan HYEON, Seoul (KR); Hyekjin KWON, Seoul (KR); Dokyoon KIM, Seoul (KR)

(21) Appl. No.: 17/253,068

(22) PCT Filed: Jun. 17, 2019
(86) PCT No.: PCT/KR2019/007262

§ 371 Date: Dec. 16, 2020

(30) Foreign Application Priority Data
Jun. 22, 2018 (KR) 10-2018-0072332

(51) Int. Cl. A61K 9/51; C07K 16/18; A61K 47/69

(52) U.S. Cl. CPC A61K 9/513; A61K 47/69; C07K 16/18

(57) ABSTRACT

A nanoparticle structure is provided. The nanoparticle structure comprises a core comprising first nanoparticles and a shell located on a surface of the core and comprising second nanoparticles. The first nanoparticles may comprise magnetic nanoparticles, and the second nanoparticles may comprise catalytic nanoparticles.

### TECHNICAL FIELD

The present invention relates to a nanoparticle structure.

### BACKGROUND ART

The neuropathological role of amyloid-β in Alzheimer's disease (AD) became a major focus of Alzheimer's research since amyloid-β plaque was first observed in the postmortem brain of an Alzheimer's patient. The amyloid-β accumulation in a brain leads to nerve cell dysfunction and the formation of age spots associated with death, and the rise of amyloid-β peptides has been regarded as the main cause of the pathogenesis of Alzheimer's disease. Therefore, extensive studies for reducing these amyloid-β deposits in the brain have been conducted by immunizations produced or administered to the bloodstream of AD patients in order for a specific amyloid-β antibody to act as a peripheral amyloid-β sink or activate microglial phagocytosis of amyloid-β plaque. However, the previous amyloid-β immunotherapy has problems causing unwanted side effects such as meningitis and microhaemorrhage. Therefore, the development of clinical related technology for reducing amyloid-β from AD patients is not progressing.

### DISCLOSURE

### Technical Problem

In order to solve the above mentioned problems, the present invention provides a new nanoparticle structure.

The present invention provides a nanoparticle structure that can be used to treat disease without side effects.

The other objects of the present invention will be clearly understood by reference to the following detailed description and the accompanying drawings.

### Technical Solution

A nanoparticle structure according to an embodiment of the present invention comprises a core comprising first nanoparticles and a shell located on a surface of the core and comprising second nanoparticles.

The first nanoparticles may comprise magnetic nanoparticles. The first nanoparticles may comprise first metal oxide nanoparticles. The first metal oxide nanoparticles may comprise iron oxide nanoparticles.

The second nanoparticles may comprise catalytic nanoparticles. The second nanoparticles may comprise second metal oxide nanoparticles. The second metal oxide nanoparticles may comprise cerin nanoparticles.

The nanoparticle structure may further comprise an antibody combined to the second nanoparticles. The antibody may comprise an amyloid-β antibody. The antibody may be combined to the second nanoparticles by polyacrylic acid.

The nanoparticle structure may further comprise a dispersible compound combined to the second nanoparticles. The dispersible compound may comprise PEG.

The core may comprise a cluster where a plurality of the first nanoparticles are assembled. The nanoparticle structure may have a hydrodynamic diameter of 200˜400 nm.

### Advantageous Effects

A nanoparticle structure according to the embodiments of the present invention and a blood cleansing system using the nanoparticle structure can be easily used to treat various diseases such as AD. The nanoparticle structure can specifically capture amyloid-β peptide from blood with high capture efficiency, and can be easily retrieved from blood by magnetic separation. The nanoparticle structure is injected into blood in vitro by the blood cleansing system to conduct blood cleansing and is not injected into the body. The nanoparticle structure and the blood cleansing system do not cause side effects such as oxidative stress, infection, cardiovascular disease and the like, and are advantageous to patients since WBC, RBC, PLT, NEU, MCV and MPV values do not change significantly. In addition, oxidative stress can be reduced and inflammation can be prevented since it is possible to remove a large amount of reactive oxygen species of various types during blood cleansing.

### DESCRIPTION OF DRAWINGS

FIG. 1 shows an iron oxide/ceria nanoparticle structure according to an embodiment of the present invention.

FIG. 2 schematically shows a process of forming the iron oxide/ceria nanoparticle structure of FIG. 1.

FIG. 3 is a view for explaining the versatility of the iron oxide/ceria nanoparticle structure of FIG. 1.

FIG. 4 shows a TEM image of iron oxide nanoparticles.

FIG. 5 shows a TEM image of an iron oxide nanoparticle cluster.

FIG. 6 shows a TEM image of ceria nanoparticles.

FIG. 7 shows a TEM image of an iron oxide/ceria nanoparticle structure.

FIG. 8 shows a magnetization curve of an iron oxide/ceria nanoparticle structure measured at a temperature of 300K.

FIG. 9 shows SOD-mimetic activity of an iron oxide/ceria nanoparticle structure according to ceria concentration in comparison with ceria nanoparticles.

FIG. 10 shows CAT-mimetic activity of an iron oxide/ceria nanoparticle structure according to ceria concentration in comparison with ceria nanoparticles.

FIG. 11 shows a blood cleansing system according to an embodiment of the present invention.

FIG. 12 shows changes in amyloid-β in plasma before and after blood cleansing treatment using an iron oxide/ceria nanoparticle structure.

FIG. 13 shows the ROS (reactive oxygen species) level in plasma after blood cleansing treatment using an iron oxide/ceria nanoparticle structure.

FIG. 14 shows the concentration ratio of amyloid-β/GAPDH in a mouse brain after blood cleansing treatment using an iron oxide/ceria nanoparticle structure.

FIG. 15 shows the plaques level of amyloid-β in a mouse brain after blood cleansing treatment using an iron oxide/ceria nanoparticle structure.

FIG. 16 shows the manifestation level of GFAP in a mouse brain after blood cleansing treatment using an iron oxide/ceria nanoparticle structure.

FIG. 17 is CLSM (confocal laser scanning microscopy) images showing the amyloid-β of FIG. 15 and the GFAP manifestation of FIG. 16.

FIGS. 18 to 23 show WBC, RBC, PLT, NEW, MCV, and MPV in mouse blood after blood cleansing treatment using an iron oxide/ceria nanoparticle structure, respectively.

### BEST MODE

Hereinafter, a detailed description will be given of the present invention with reference to the following embodiments. The purposes, features, and advantages of the present invention will be easily understood through the following embodiments. The present invention is not limited to such embodiments, but may be modified in other forms. The embodiments to be described below are nothing but the ones provided to bring the disclosure of the present invention to perfection and assist those skilled in the art to completely understand the present invention. Therefore, the following embodiments are not to be construed as limiting the present invention.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

When it is mentioned that an element is bonded to another element, it means that it may be directly bonded to the other element or a third element may be interposed between them.

The size of the element or the relative sizes between elements in the drawings may be shown to be exaggerated for more clear understanding of the present invention. In addition, the shape of the elements shown in the drawings may be somewhat changed by variation of the manufacturing process or the like. Accordingly, the embodiments disclosed herein are not to be limited to the shapes shown in the drawings unless otherwise stated, and it is to be understood to comprise a certain amount of variation.

The term of A/B nanoparticle structure used herein means a nanoparticle structure having a core (A)—shell (B) structure where a plurality of B nanoparticles are disposed on the surface of A nanoparticle or the surface of a cluster where a plurality of A nanoparticles are assembled. For example, an iron/ceria nanoparticle structure means a nanoparticle structure where a cluster where a plurality of iron oxide nanoparticles are assembled is a core and a plurality of ceria nanoparticles disposed on the surface of the cluster is a shell. The B (shell) may partially cover the surface of the A (core) or may cover the entire surface.

A nanoparticle structure according to embodiments of the present invention may comprise a core comprising first nanoparticles, a shell located on a surface of the core and comprising second nanoparticles, a dispersible compound combined to the second nanoparticles, and an antibody.

The first nanoparticles may comprise magnetic nanoparticles. The first nanoparticles may comprise first metal oxide nanoparticles. The first metal oxide nanoparticles may comprise iron oxide nanoparticles. The core may comprise a cluster where a plurality of the first nanoparticles are assembled.

The second nanoparticles may comprise catalytic nanoparticles. The second nanoparticles may comprise second metal oxide nanoparticles. The second metal oxide nanoparticles may comprise ceria nanoparticles.

The antibody and the dispersible compound may be combined to the second metal oxide nanoparticles by polyacrylic acid.

The antibody may comprise an amyloid-β antibody. The dispersible compound may comprise PEG.

The nanoparticle structure may have a hydrodynamic diameter of 200˜400 nm.

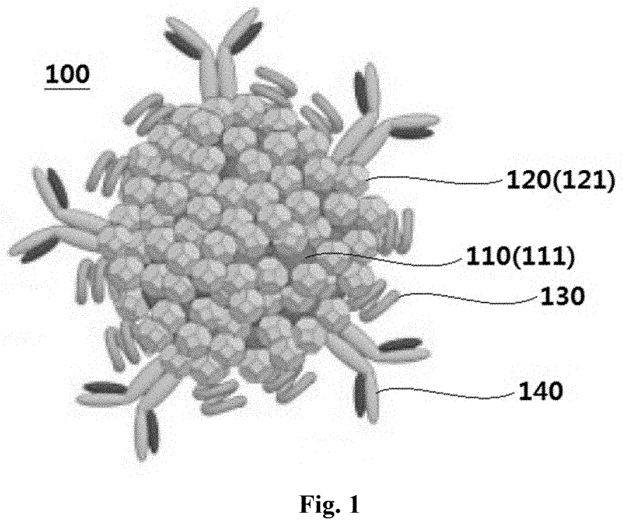

FIG. 1 shows an iron oxide/ceria nanoparticle structure according to an embodiment of the present invention.

Referring to FIG. 1, the iron oxide/ceria nanoparticle structure 10 may comprise a core 110, a shell 120, a dispersible compound 130 and an antibody 140.

The core 110 may comprise iron oxide nanoparticles 111. For example, the core 110 may comprise a cluster in which a plurality of iron oxide nanoparticles 111 are assembled. The core 110 may have magnetic properties so that it can separate the iron oxide/ceria nanoparticle structure 10 from blood after blood cleansing treatment. The iron oxide nanoparticles 111 may have a diameter of about 10 nm, and the core 110 may have a diameter of about 200 nm.

The shell 120 may comprise ceria nanoparticles 121 located on the surface of the core 110. In addition, the shell 120 may be formed of a single layer of the ceria nanoparticles 121. The ceria nanoparticles 121 can remove reactive oxygen species during blood cleansing treatment. The ceria nanoparticle 121 may have a diameter of about 3 nm.

The dispersible compound 130 may be combined to the ceria nanoparticles 121 to provide dispersibility to the iron oxide/ceria nanoparticle structure 100. The dispersible compound 130 may have water dispersibility and/or oil dispersibility. For example, the dispersible compound 130 may comprise PEG (polyethylene glycol) or Lipid-PEG. The dispersible compound 130 may be combined to the ceria nanoparticles 121 by polyacrylic acid.

The antibody 140 is combined to the ceria nanoparticles 121 and can be used for various blood cleansing. For example, the antibody 140 may comprise an amyloid-β antibody and can be used to treat Alzheimer's disease. The antibody 140 may be combined to the ceria nanoparticles 121 by polyacrylic acid.

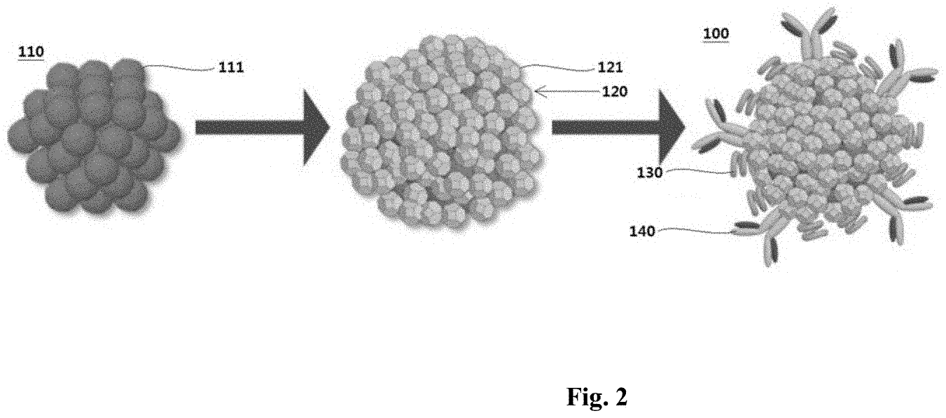

FIG. 2 schematically shows a process of forming the iron oxide/ceria nanoparticle structure of FIG. 1.

Referring to FIG. 2, iron oxide nanoparticles 111 are formed. Iron oxide nanoparticles 111 can be synthesized by thermal decomposition of iron-oleate complex. Iron chloride (III) (10.8 g, Aldrich, 97%) and sodium oleate (36.5 g, TCI, 97%) are dissolved in a mixture of 80 ml ethanol, 60 ml deionized water and 140 ml hexane. After the reaction of this mixture solution is performed at 60° C. for 8 hours, it is cooled to room temperature. After separating an upper organic layer, it is washed 3 times with deionized water. The iron-oleate complex can be obtained by evaporating hexane from the separated organic solution. The mixture solution of iron-oleate (1.8 g), oleic acid (0.28 g, Aldrich, 90%) and 1-octadecene (12 g, Aldrich, 90%) is heated at 320° C. (heating rate is 1° C./min). After conducting an aging step for 30 minutes with vigorous stirring at this temperature, the mixture solution is cooled to room temperature. As a result, iron oxide nanoparticles 111 having a size of about 10 nm are formed. The iron oxide nanoparticles 111 are washed with an excess of ethanol and then refined by centrifugation. After repeating the washing and centrifugation steps 2 more times, the obtained iron oxide nanoparticles 111 are dispersed in chloroform.

Ceria nanoparticles 121 are formed. The mixture solution of cerium (III) acetate (0.32 g, Aldrich, 99.9%), oleyl amine (3.2 g, Acros, 85%) and xylene (13 g) is stirred vigorously for 12 hours at room temperature. The mixture solution is heated at a heating rate of 2° C./min. Deionized water (1 g) is rapidly injected into the mixture solution at 90° C. The mixture solution is maintained at this temperature for 3 hours and then cooled to room temperature. As a result, ceria nanoparticles 121 having a size of about 3 nm are formed. After washing the solution with an excess of ethanol, ceria nanoparticles 121 are separated by centrifugation. After repeating the washing and centrifugation steps 2 more times, the obtained ceria nanoparticles 121 are dispersed in chloroform.

The iron oxide/ceria nanoparticle structure 100 is formed by coating the ceria nanoparticles 121 on the self-assembled cluster 110 of the iron oxide nanoparticles 111. By mixing iron oxide nanoparticles (150 mg) in chloroform (4.5 g) with dodecyltrimethylammonium bromide (150 mg, Aldrich, 98%) in deionized water (10 g) while vigorously stirring them, the iron oxide nanoparticle cluster 110 with a size of about 200 nm where the iron oxide nanoparticles 111 are assembled is formed. The chloroform is evaporated and the solution is mixed with polyacrylic acid (0.9 g, Aldrich) in ethylene glycol (11.1 g, Aldrich, 99.8%). After conducting washing with an excess of deionized water, the iron oxide nanoparticle cluster 110 is separated by centrifugation. In order to coat the iron oxide nanoparticle cluster 110 with the ceria nanoparticles 121, the iron oxide nanoparticle cluster 110 is mixed with the ceria nanoparticles 121 in chloroform that are separately produced. After stirring them overnight, the iron/ceria nanoparticle structure 100 is separated by centrifugation. The iron oxide/ceria nanoparticle structure 100 is dispersed in deionized water to be mixed with polyacrylic acid (0.9 g) and stirred for 1 hour, and then excess polyacrylic acid is removed by centrifugation.

The antibody 140 is comprised by being combined to the iron oxide/ceria nanoparticle structure 100 through a covalent bond between the polyacrylic acid and the antibody. 1-ethyl-3-(3-dimethylaminopropyl)urea hydrochloride (1 mg, Aldrich, 99%) and N-hydroxysuccinimide (1 mg, Aldrich, 98%) in 2-(N-morpholino) ethanesulfonic acid buffer solution (100 μL, pH 4.7) are added to the iron oxide/ceria nanoparticle structure 100 (1 mg[Fe]) dispersed in deionized water (1 mL) and kept at constant temperature for 30 minutes. Then the iron oxide/ceria nanoparticle structure 100 is separated by centrifugation and is mixed with an anti-human amyloid-β antibody (500 μL, BioLegend, 800702).

After 1 hour, the iron oxide/ceria nanoparticle structure 100 combined with the antibody is separated by centrifugation and dispersed in borate buffer solution. PEG (2000)-amine (50 mg) in phosphate buffer solution (PBS) is added to the solution and kept at constant temperature for 2 hours so that PEG 130 is combined with the iron oxide/ceria nanoparticle structure 100. The iron oxide/ceria nanoparticle structure 100 is separated by centrifugation after conducting washing, dispersed in phosphate buffer solution (500 μL), and stored at 4° C. before using it.

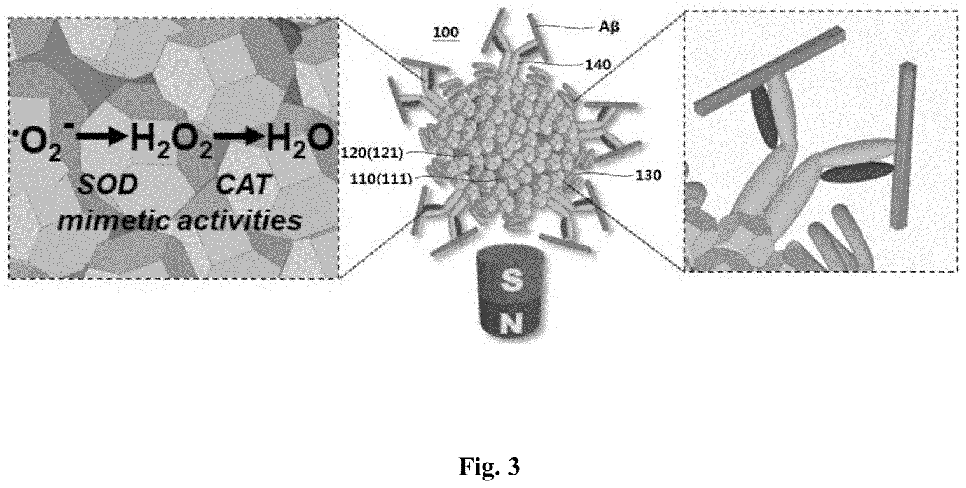

FIG. 3 is a view for explaining the versatility of the iron oxide/ceria nanoparticle structure of FIG. 1.

Referring to FIG. 3, the iron oxide nanoparticles 111 assembled at the core 110 of the iron oxide/ceria nanoparticle structure 100 generate a large magnetic adsorptive force toward an outer magnet to make possible the separation of amyloid-β peptide (Aβ) captured by the antibody 140. The ceria nanoparticles 121 in the shell 120 of the iron oxide/ceria nanoparticle structure 100 exert a regeneration catalyst action for removing ROS (reactive oxygen species) produced by an immune response in the course of the reaction (blood cleansing). Blood amyloid-β cleansing treatment on 5XFAD transgenic mouse reduces the amount of amyloid-β plaques in the brain to make it possible to prevent and treat Alzheimer's disease.

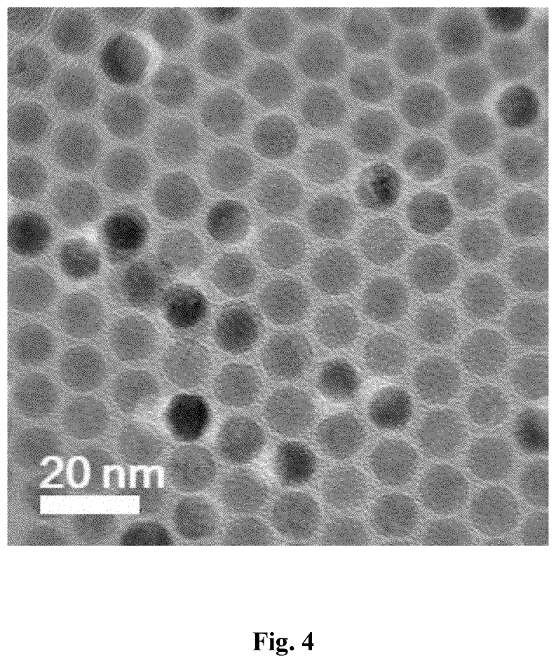

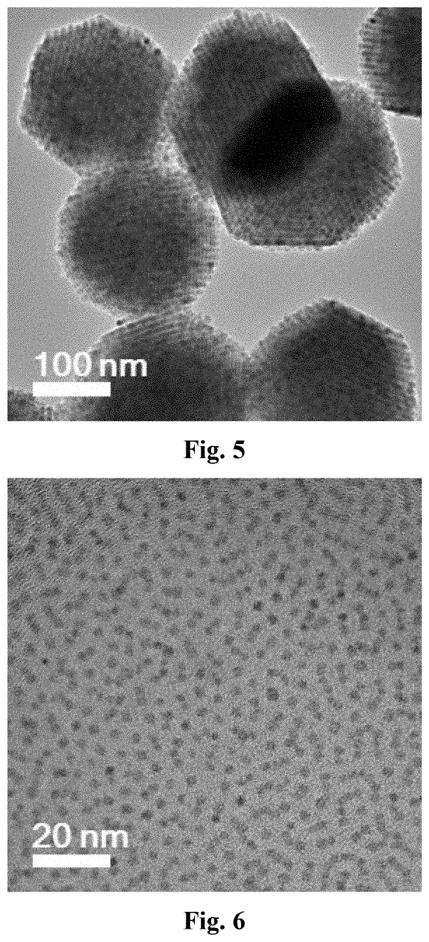

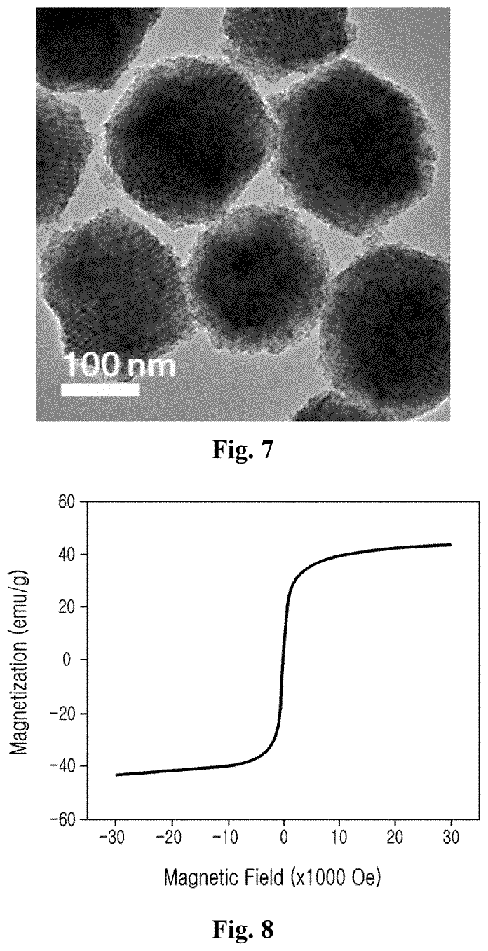

FIG. 4 shows a TEM image of iron oxide nanoparticles, FIG. 5 shows a TEM image of an iron oxide nanoparticle cluster, FIG. 6 shows a TEM image of ceria nanoparticles, and FIG. 7 shows a TEM image of an iron oxide/ceria nanoparticle structure.

Referring to FIGS. 4 to 7, iron oxide nanoparticles having a size of about 10 nm are synthesized by pyrolysis of iron oleate complex (FIG. 4). A TEM (Transmission electron microscopy) image shows a superlattice structure of self-assembly due to the size uniformity of the iron oxide nanoparticles (FIG. 5). FIG. 6 shows ceria nanoparticles having a size of about 3 nm, and the ceria nanoparticles are coated on the cluster of the iron oxide nanoparticles (FIG. 7). The layer of the ceria nanoparticles has a thickness of about 3 nm, which means that a single layer of ceria nanoparticles covers the iron oxide nanoparticle cluster.

The core/shell structure assembly of the iron oxide/ceria nanoparticle structure is coated with polyacrylic acid for the structure stabilization and the covalent bond between amyloid-β antibody and anti-fouling polyethylene glycol (PEG). The formed iron oxide/ceria nanoparticle structure has a hydrodynamic diameter of about 250 nm and a ζ-potential value of −45 mV. After the bonding of the antibody and PEG, the hydrodynamic diameter and ζ-potential value increase to about 330 nm and −23 mV, respectively.

The antibody of the iron oxide/ceria nanoparticle structure can be identified by SDS-PAGE (sodium dodecyl sulfate-polyacrylamide gel electrophoresis) and is stably maintained in the iron oxide/ceria nanoparticle structure without noticeable decrease in activity for at least 1 month.

FIG. 8 shows a magnetization curve of an iron oxide/ceria nanoparticle structure measured at a temperature of 300 K.

Referring to FIG. 8, the magnetization curve of the iron oxide/ceria nanoparticle structure measured at room temperature does not show any hysteresis loop predictable from super paramagnetic materials.

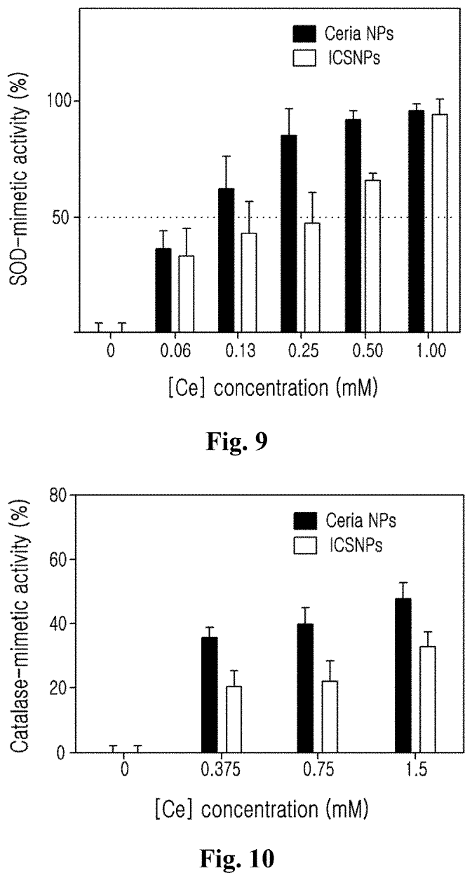

FIG. 9 shows SOD-mimetic activity of an iron oxide/ceria nanoparticle structure according to ceria concentration in comparison with ceria nanoparticles, FIG. 10 shows CAT-mimetic activity of an iron oxide/ceria nanoparticle structure according to ceria concentration in comparison with ceria nanoparticles.

Referring to FIGS. 9 and 10, ROS (reactive oxygen species) scavenging activity of the iron oxide/ceria nanoparticle structure (ICSNPs) is evaluated by using SOD (superoxide dismutase) and CAT (catalase) activity analysis. The dose-dependent activity observed in both analyses clearly shows the ability of the iron oxide/ceria nanoparticle structure of removing peroxide (O²⁻) and hydrogen peroxide (H₂O₂) by using SOD and CAT mimetic activities of the ceria nanoparticle shell, respectively. The low reactive oxygen species scavenging ability of the iron oxide/ceria nanoparticle structure compared to the individually dispersed ceria nanoparticles may result from the low surface-to-volume ratio.

Although not shown in the drawing, in the MTT analysis performed to evaluate the cytotoxicity of the iron oxide/ceria nanoparticle structure on HeLa cells, cell viability is maintained largely at a high concentration of the iron oxide/ceria nanoparticle structure of 1.0 mM[Fe]. Like this, the iron oxide/ceria nanoparticle structure can have good biocompatibility by PEG coating, and cellular absorption of the iron oxide/ceria nanoparticle structure can be prevented because of its large size.

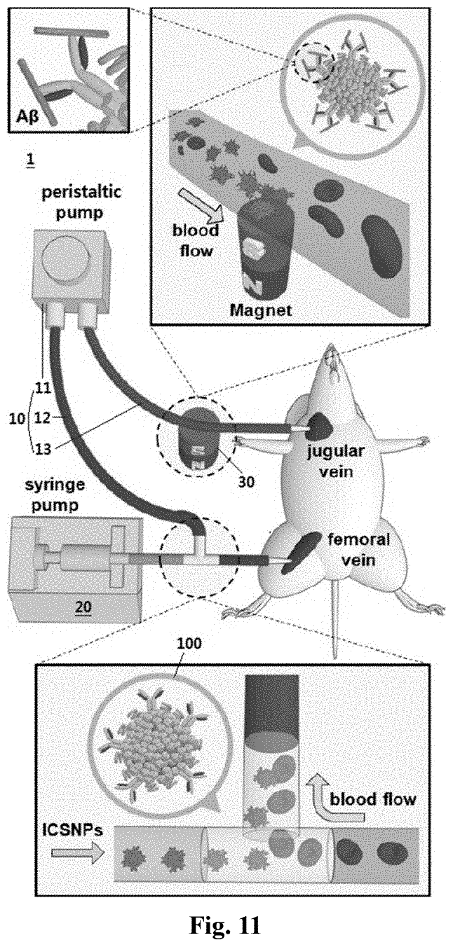

FIG. 11 shows a blood cleansing system according to an embodiment of the present invention.

Referring to FIG. 11, the blood cleansing system 1 comprises a blood circulation part 10, a nanoparticle structure supply part 20 and a nanoparticle structure recovery part 30.

The blood circulation part 10 may comprise a blood circulation pump 11, a blood discharge tube 12 and a blood injection tube 13. The blood circulation pump 11 is disposed between the blood discharge tube 12 and the blood injection tube 13. The blood circulation pump 11 discharges blood to the outside of the body and then injects it back into the body to circulate blood. The blood circulation pump 11 may comprise, for example, a peristaltic pump. The blood discharge tube 12 is disposed between a point of discharging blood and the blood circulation pump 11 to discharge blood from the body to the outside of the body. The blood injection tube 13 is disposed between a point of injecting blood and the blood circulation pump 11 to inject blood treated with the blood cleansing to the body.

The nanoparticle structure supply part 20 is connected to the blood circulation part 10 to supply the nanoparticle structure 100 to blood discharged from the body. The nanoparticle structure supply part 20 may comprise, for example, a syringe pump. The nanoparticle structure supply part may supply the nanoparticle structure 100 to the blood discharge tube 12 adjacent to a point of discharging blood.

The nanoparticle structure recovery part 30 is connected to the blood circulation part 10 to retrieve the nanoparticle structure 100 that captured amyloid-β peptide (Aβ) from the blood cleansing. The nanoparticle structure recovery part 30 may comprise, for example, a permanent magnet. The nanoparticle structure supply part 30 can retrieve the nanoparticle structure 100 from the blood injection tube 13 adjacent to a point of injecting blood.

FIG. 11 schematically shows one example of conducting blood amyloid-β cleansing treatment by using 5XFAD transgenic mouse as a model of Alzheimer's disease and using an iron oxide/ceria nanoparticle structure (ICSNPs).

Referring again to FIG. 11, a peristaltic pump discharges blood from a femoral vein of an anesthetized mouse and circulates it in vitro at a flow rate of 150 μL/min. The iron oxide/ceria nanoparticle structure solution (1.8 mM[Fe]) is injected by the syringe pump working at a flow rate of 10 μL/min in the vicinity of a start point of an extracorporeal circulation circuit via a micromixer. The iron oxide/ceria nanoparticle structure is used after being diluted in a solution at an appropriate concentration in consideration of the efficiency of capturing amyloid-β peptide within a concentration range that does not have cytotoxicity in the MTT analysis result. Blood discharged from the body is mixed with the iron oxide/ceria nanoparticle structure where amyloid-β antibody is combined in the extracorporeal circulation circuit, and the amyloid-β antibody specifically captures the amyloid-β peptide in blood.

The permanent magnet is located near the end of the circuit to separate the iron oxide/ceria nanoparticle structure and the amyloid-β peptide combined to it from blood. The core of the iron oxide/ceria nanoparticle structure is made up of a plurality of self-assembled superparamagnetic iron oxide nanoparticles, and can be magnetically separated together with the captured amyloid-β peptide by applying an external magnetic field. The ceria nanoparticles existing at the shell of the iron oxide/ceria nanoparticle structure can remove reactive oxygen species (ROS) that may be generated when blood meets foreign substances.

The separated iron oxide/ceria nanoparticle structure remains in a tube without blocking the flow channel to be retrieved. The treated blood is magnetically separated and then injected into the jugular vein of the mouse to return to the bloodstream.

Since the amyloid-β peptide-antibody complex and the remaining unused amyloid-β antibody are not injected into the body of the mouse, a subsequent treatment on them in the body is not needed.

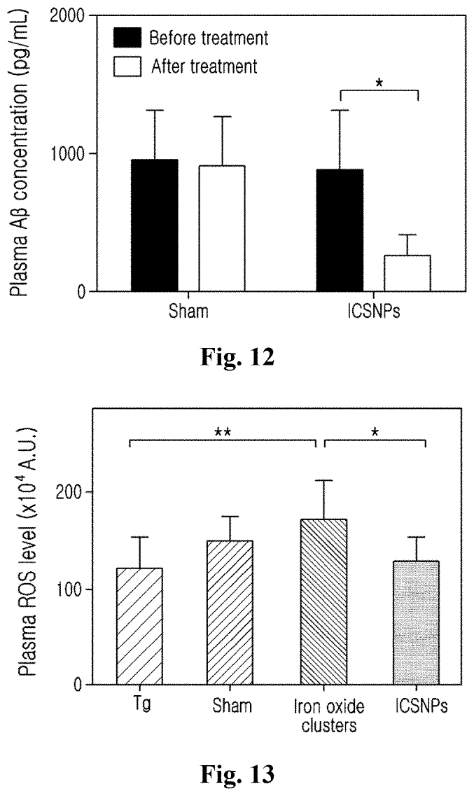

FIG. 12 shows changes in amyloid-β in plasma before and after blood cleansing treatment using an iron oxide/ceria nanoparticle structure (ICSNPs).

Referring to FIG. 12, the performance of blood amyloid-β cleansing is evaluated by comparing the measured amyloid-β peptide concentrations before and after the cleansing treatment. A total of 20 plasma samples before and after sham treatment or iron oxide/ceria nanoparticle structure treatment (5 per group) are obtained from a two-month-old 5XFAD transgenic mouse, and the amyloid-β level of them is analyzed. It shows that 76% of amyloid-β peptides in blood are removed on average by the iron oxide/ceria nanoparticle structure treatment. On the other hand, regarding the sham group where the iron oxide/ceria nanoparticle structure is not used during the blood treatment, there is no significant difference between the plasma amyloid-β levels measured before and after the treatment. Although not shown in the drawing, Western blot data also shows a decrease in plasma amyloid-β level after the cleansing treatment.

FIG. 13 shows the ROS (reactive oxygen species) level in plasma after blood cleansing treatment using an iron oxide/ceria nanoparticle structure (ICSNPs).

Referring to FIG. 13, the ceria nanoparticle shell of the iron oxide/ceria nanoparticle structure helps to suppress the generation of reactive oxygen species during the blood cleansing treatment. If the nanoparticle structure of the present invention is not used or ultra-nanoparticles composed of only iron oxide nanoparticles are used when conducting the blood cleansing, the reactive oxygen species level in plasma increases significantly. The increase in the reactive oxygen species level can be minimized by using the iron oxide/ceria nanoparticle structure.

The effect of the blood amyloid-β cleansing treatment on the amount of amyloidβ in the brain is investigated by using immunostaining of brain sections. Two-month-old 5XFAD transgenic mice are divided into 3 groups (5 per group). The first group (untreated group, Tg) does not receive the blood cleansing treatment. The second group (treated group, Treatment) receives the blood cleansing treatment twice at one month intervals using the iron oxide/ceria nanoparticle structure. The last group (sham group, Sham) also receives the blood cleansing treatment twice, but the iron oxide/ceria nanoparticles are not used. All mice are sacrificed 4 months after, and brains are separated and analyzed. Since each brain sample is obtained from different mice, this is different from the previously described experiment of comparing plasma amyloid-β levels where each sample set before and after the blood cleansing treatment is obtained in the same mouse, but a general trend can still be observed in the data.

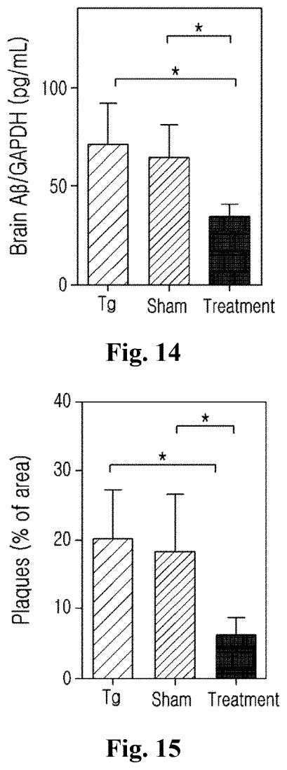

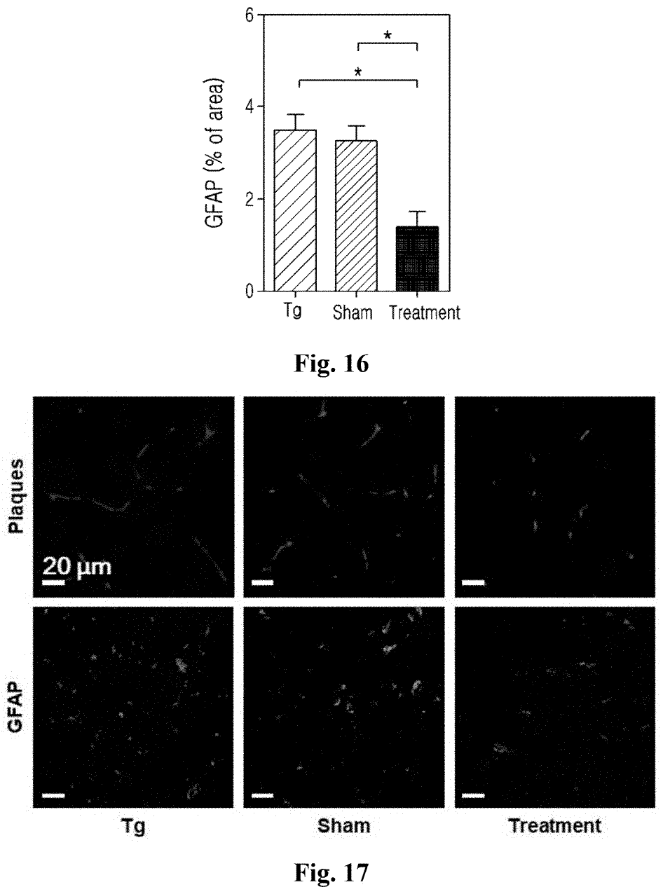

FIG. 14 shows the concentration ratio of amyloid-β/GAPDH in a mouse brain after blood cleansing treatment using an iron oxide/ceria nanoparticle structure, FIG. 15 shows the plaques level of amyloid-β in a mouse brain after blood cleansing treatment using an iron oxide/ceria nanoparticle structure, FIG. 16 shows the manifestation level of GFAP in a mouse brain after blood cleansing treatment using an iron oxide/ceria nanoparticle structure, and FIG. 17 is CLSM (confocal laser scanning microscopy) images showing the amyloid-β of FIG. 15 and the GFAP manifestation of FIG. 16.

Referring to FIG. 14, the brain amyloid-β level in the treated group is significantly lower than the brain amyloid-β level in the untreated group. On the contrary, there is no significant difference between the brain amyloid-β level in the untreated group and the brain amyloid-β level in the sham group.

Referring to FIG. 15, the result of immunohistofluorescence analysis regarding coronal sections of mouse brains (5 mice per group) is similar to the immunoassay result. That is, the level of amyloid-β plaques in the cerebral cortex in the treated group decreases significantly when compared to the untreated group, and there is no significant difference between the untreated group and the sham group.

Referring to FIG. 16, the GFAP (Glial fibrillary acidic protein) manifestation of cerebral astrocyte which is related to neuroinflammation, also shows a significant decrease in the treated group.

Referring to FIG. 17, the results of FIGS. 14 to 16 can also be confirmed in CLSM images.

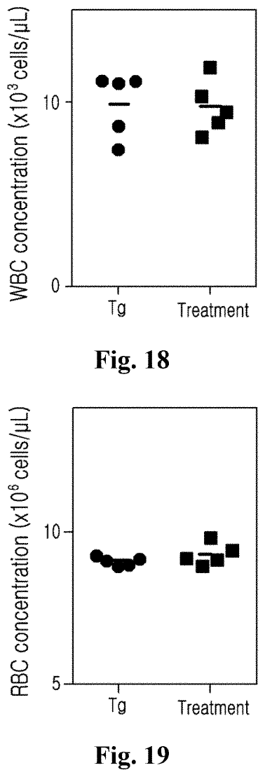

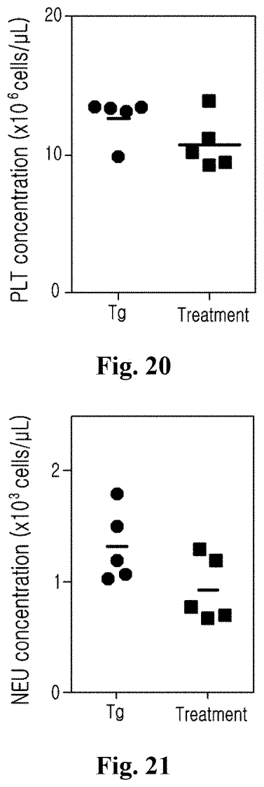

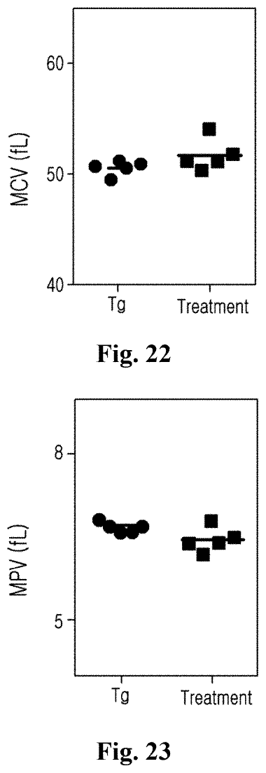

FIGS. 18 to 23 show WBC, RBC, PLT, NEW, MCV, and MPV in mouse blood after blood cleansing treatment using an iron oxide/ceria nanoparticle structure, respectively. Blood samples obtained from sacrificed mice are analyzed to evaluate the composition change.

Referring to FIGS. 18 to 20, the result of complete blood count (CBC) shows there is no significant difference between the treated group and the untreated group in the concentration of white blood cell (WBC), red blood cell (RBC) and platelet (PLT).

Referring to FIGS. 21 to 23, there is no significant difference between the treated group and the untreated group in neutrophil (NEU) concentrations, mean corpuscular volumes (MCV) and mean platelet volumes (MPV) that are important parameters in evaluating the function of WBC, RBC and PLT. This indicates that the blood cleansing treatment using the iron oxide/ceria nanoparticle structure does not cause mice serious inflammation or side effects.

### Analysis Example

SOD and CAT-Mimetic Activity Analysis

SOD-mimetic activity is measured using SOD assay kits (Sigma-Aldrich, 19160). The iron oxide/ceria nanoparticle structure is included in 600 μL of WST-1 (water-soluble tetrazolium salt; 2-(4-iodophenyl)-3-(4-nitrophenyl)-5-(2,4-disulfo-phenyl)-2H-tetrazolium monosodium salt) solution at concentrations of 0, 0.06, 0.125, 0.25, 0.5 and 1 mM[Ce]. The prepared solution of the iron/ceria nanoparticle structure is transferred to microplate wells three times (each 200 μL). After adding xanthine oxidase (20 μL) to each well, the microplate is kept at constant temperature of 37° C, for 20 minutes. SOD-mimetic activity is measured by measuring the absorbance of each well at 450 nm, and 50 U/mL SOD is defined as the amount of SOD inhibiting the reduction reaction of WST-1 by 50%.

CAT-mimetic activity is measured using CAT assay kits (Amplex Red hydrogen peroxide/peroxidase assay kit, Molecular Probes, A22188). The iron oxide/ceria nanoparticle structure is diluted to a reaction buffer solution containing 100 μM Amplex Red reagent and 2 mM hydrogen peroxide at different concentrations (0, 0.375, 0.75 and 1.5 mM[Ce]). 50 μL of each solution is transferred to microplate wells three times. After keeping constant temperature at room temperature for 30 minutes, the absorbance of each well at 490 nm is measured. 1 mU/mL HRP (horseradish peroxidase) is used as a 100% control group when measuring CAT-mimetic activity.

Cell Culture

HeLa cells are cultured in DMEM (Dulbecco's modified Eagle's medium) supplemented with inactivation FBS (fetal bovine serum) heated by 10%. Cells are maintained at 1×10⁵ cells/mL at 37° C. under a humidification atmosphere of 5% CO₂.

Cell Viability Analysis

HeLa cells are inoculated into 96-well plates (10,000 cells/well) and cultured for 12 hours. The iron oxide/ceria nanoparticle structure diluted in a cell culture medium is added to each microplate well by 100 μL to make final concentrations 0, 0.06, 0.125, 0.25, 0.5 and 1 mM[Fe] (0, 2.38, 4.75, 9.5, 19 and 38 μM[Ce], respectively). The cells are cultured at 37° C. for 24 hours, and 20 μL MTT (3-[4,5-dimethylthialzol-2-yl]-2,5-diphenyltetrazolium bromide) (5 mg/mL) is added to each well. After culturing the cells at 37° C. for 4 hours, dimethyl sulfoxide (200 μL) is added to each well. Cell viability is determined by measuring the absorbance of each well at 595 nm.

Blood Amyloid-β Peptide Cleansing

A two-month-old 5XFAD transgenic male mouse is anesthetized with isofluorane. 29 gauge needle connected to a medical tube (inner diameter=0.5 mm) is inserted into the femoral vein of the mouse and used as a blood discharge point. The inner side of the needle and the tube is pretreated with 2% heparin in PBS for 8 hours to prevent blood clotting during the cleansing treatment.

Blood circulation through the tube is initiated at a flow rate of 150 μL/min by a peristaltic pump. At the same time, PBS solution of the iron oxide/ceria nanoparticle structure (1.8 mM[Fe]) is introduced into blood at a flow rate of 10 μL/min by a syringe pump through a three-way micromixer closely connected to the blood discharge point. The other end of the tube is connected to 31 gauge needle inserted into the jugular vein of the mouse so that the treated blood is injected back into the body. A neodymium magnet is placed near the end of the extracorporeal blood circuit that is the blood injection point in order to separate the iron oxide/ceria nanoparticle structure and the amyloid-β peptide combined to it. The blood cleansing is conducted for 0.5 minutes per 1 g of mouse weight.

Plasma Amyloid-β Immunoassay

Quantification is conducted using human amyloid-β enzyme-linked immunosorbent assay (ELISA) kits (R&D systems, DAB142). Blood samples from each mouse are collected by microtubes treated with anticoagulants before and after blood cleansing treatment (each about 50 μL). After removing cells by centrifugation, the generated supernatant is diluted tenfold with a diluent buffer solution (RD2-7) and then analyzed. Measurements are performed 3 times.

Western Blot

Plasma samples obtained before and after blood cleansing treatment are diluted 1,000-fold with PBS. Samples are denatured at 97° C. for 5 minutes and cooled in ice for 10 minutes. After centrifugation, 10 μl of each supernatant is loaded onto SDS-PAGE gel. The gel is transferred onto a nitrocellulose membrane. The membrane is washed for one hour with 5% skim milk in 0.1M PBS containing 0.05% Tween 20 (PBST) and then is blocked.

It reacts overnight with anti-amyloid-β antibody (BioLegend, 800702) diluted with PBST containing 5% skim milk (1:1000). After conducting washing with 5% skim milk in PBST, HRP-coupled goat polyclone anti-mouse antibody (Abeam, ab6789) is used as secondary antibody. After conducting a rinse, HRP substrate reagent (Merck Millipore, WBLUR0500) is applied for 2 minutes at room temperature. Chemiluminescent signals are captured for analysis.

Plasma Reactive Oxygen Species Analysis

Reactive oxygen species levels are compared using ROS/RNS assay kits (Cell Biolabs, STA-347). Plasma samples obtained before and after blood cleansing treatment are diluted 100 times with PBS, and 50 μL of each sample is transferred to 96-well microplate. 50 μl of a diluted catalyst (Part No. 234703) solution is added to each well. After maintaining a constant temperature for 5 minutes at room temperature, 100 μL of a diluted dichlorodihydrofluorescein (Part No. 234704) solution is added to each well to maintain a constant temperature for 15 minutes. Fluorescence at 530 nm is measured using 480 nm excitation. Measurements are performed 3 times.

Brain Amyloid-β Immunoassay

A two-month-old 5XFAD transgenic male mouse receives blood amyloid-β peptide cleansing treatment. The mouse is grown in the same sterile laboratory condition for one month before receiving second blood amyloid-β peptide cleansing treatment on other parts of the body.

The mouse is grown for one more month and then euthanized with CO₂. Blood is collected in anticoagulant-treated CBC tubes by myocardial infarction for blood analysis. The mouse is perfused with 4% paraformaldehyde in PBS and decapitated for brain removal. The left cerebral hemisphere of each brain is dissolved in 1.5 mL of a radioimmunoprecipitation assay (RIPA) buffer solution containing 1% protease inhibitor cocktail (Cell Biolabs, AKR-190). After centrifugation, the supernatant is divided and stored at −80° C. until use.

For quantification of amyloid-β, the brain dissolved liquid is diluted 5-fold with a dilution buffer solution (RD2-7) and analyzed using human amyloid-β ELISA kits (R&D systems, DAB142). The measured amyloid-β concentration of each brain dissolved solution is standardized about the concentration of GAPDH (glyceraldehyde 3-phosphate dehydrogenase) of the same brain dissolved solution. For the measurement of GAPDH, the brain dissolved solution is diluted 10,000 times and analyzed using GAPDH ELISA kits (R&D systems, DYC5718). All measurements are performed 3 times.

Immunohistofluorescence

The right cerebral hemisphere of the obtained brain is fixed in 0.1M phosphate buffer solution containing 4% paraformaldehyde at 4° C. for 20 hours, and then is stored in 0.05M PBS containing 30% sucrose for 72 hours at 4° C. using a cryostat before cutting it into 30 μm sections. The prepared tissue section is placed on a glass slide, immersed in acetone at −20° C. for 10 minutes, and washed with PBST. The tissue section is blocked with a blocking buffer solution (ThermoFisher, 37538) containing 0.05% Tween-20 at room temperature for 1 hour.

The tissue section is rinsed with PBST, and is maintained at constant temperature for 2 hours together with an anti-amyloid-β antibody (1:1000, BioLegend, 800702) or an anti-GFAP antibody (1:200, ThermoFisher MA5-12023). After conducting washing with PBST, Alexa Fluor 594-conjugated polyclonal anti-mouse antibody (1:200, ThermoFisher R37121) is added as a secondary antibody and then is maintained at constant temperature for 1 hour. After washing the tissue section again, the fluorescence of the cerebral cortex is observed under CLSM.

Although the embodiments of the present invention have been disclosed for illustrative purposes, those skilled in the art will appreciate that the present invention may be embodied in other specific ways without changing the technical spirit or essential features thereof. Therefore, the embodiments disclosed in the present invention are not restrictive but are illustrative. The scope of the present invention is given by the claims, rather than the specification, and also contains all modifications within the meaning and range equivalent to the claims.

### INDUSTRIAL APPLICABILITY

A nanoparticle structure according to the embodiments of the present invention and a blood cleansing system using the nanoparticle structure can be easily used to treat various diseases such as AD. The nanoparticle structure can specifically capture amyloid-β peptide from blood with high capture efficiency, and can be easily retrieved from blood by magnetic separation. The nanoparticle structure is injected into blood in vitro by the blood cleansing system to conduct blood cleansing and is not injected into the body. The nanoparticle structure and the blood cleansing system do not cause side effects such as oxidative stress, infection, cardiovascular disease and the like, and are advantageous to patients since WBC, RBC, PLT, NEU, MCV and MPV values do not change significantly. In addition, oxidative stress can be reduced and inflammation can be prevented since it is possible to remove a large amount of reactive oxygen species of various types during blood cleansing.

1. A nanoparticle structure comprising:
a core comprising first nanoparticles; and
a shell located on a surface of the core and comprising second nanoparticles.

2. The nanoparticle structure of claim 1, wherein the first nanoparticles comprise magnetic nanoparticles.

3. The nanoparticle structure of claim 1, wherein the first nanoparticles comprise first metal oxide nanoparticles.

4. The nanoparticle structure of claim 3, wherein the first metal oxide nanoparticles comprise iron oxide nanoparticles.

5. The nanoparticle structure of claim 1, wherein the second nanoparticles comprise catalytic nanoparticles.

6. The nanoparticle structure of claim 1, wherein the second nanoparticles comprise second metal oxide nanoparticles.

7. The nanoparticle structure of claim 6, wherein the second metal oxide nanoparticles comprise ceria nanoparticles.

8. The nanoparticle structure of claim 1, further comprising an antibody combined to the second nanoparticles.

9. The nanoparticle structure of claim 8, wherein the antibody comprises an amyloid-β antibody.

10. The nanoparticle structure of claim 8, wherein the antibody is combined to the second nanoparticles by polyacrylic acid.

11. The nanoparticle structure of claim 1, further comprising a dispersible compound combined to the second nanoparticles.

12. The nanoparticle structure of claim 11, wherein the dispersible compound comprises PEG.

13. The nanoparticle structure of claim 1, wherein the core comprises a cluster where a plurality of the first nanoparticles are assembled.

14. The nanoparticle structure of claim 1, wherein the nanoparticle structure has a hydrodynamic diameter of 200˜400 nm.
